(19) United States
(12) Patent Application Publication
(10) Pub. No.: US 2009/0198110 A1
(43) Pub. Date: Aug. 6, 2009

(54) Biological Information Acquisition Telemetry System

(75) Inventors: Akinori Tamai, Tokyo (JP); Takao Ito, Tokyo (JP)

Correspondence Address: KIMBLE INTELLECTUAL PROPERTY LAW, PLLC, 1701 PENNSYLVANIA AVE., NW, SUITE 300, WASHINGTON, DC, 20006, US

(73) Assignee: NIHON KOHDEN CORPORATION, Tokyo (JP)

(21) Appl. No.: 12/363,609

(22) Filed: Jan. 30, 2009

(30) Foreign Application Priority Data
Jan. 31, 2008 (JP) 2008-020805

(51) Int. Cl. A61B 5/00

(52) U.S. Cl. 600/300

(57) ABSTRACT

A biological information acquisition telemetry system includes: a transmit device, attached to a part of a living body and configured to acquire a biological signal to transmit the biological signal as a radio signal; a relay device, configured to perform space diversity reception, the relay device including: a first receiver, configured to wirelessly receive the radio signal from the transmit device; a first transmitter, configured to transmit the radio signal received by the first receiver; and an attachment unit, adapted to hold the first receiver and the first transmitter and attach the first receiver and the first transmitter to the living body; and a center apparatus, including: a second receiver, configured to receive the radio signal from the first transmitter of the relay device; a processor, configured to generate biological information based on the radio signal received by the second receiver; and a display, configured to display the biological information generated by the processor.

### BACKGROUND OF THE INVENTION

This invention relates to a biological information acquisition telemetry system used to acquire biological information.

A related-art system is a system of the type wherein each electrode for acquiring a signal from a living body is attached to the living body, a lead wire is extended from the electrode for introducing a biological signal into a radio relay device, and the biological signal is transmitted from the radio relay device to a center apparatus.

However, in the related-art system, the electrode and the radio relay device are connected by the lead wire and thus if an attempt is made to obtain biological information of a subject in a moving state (for example, an exercise condition), the electrode is come off or contact of the electrode becomes unstable because of the length of the lead wire, and it becomes difficult to acquire stable biological information. Since the lead wire is placed so as to trail on the surface of the living body, the subject cannot do extreme exercise and insufficient biological information can only be acquired.

A system described in JP-2007-143959 is known as a related-art system solving the problem described above. In the related-art system described in JP-2007-143959, a biological signal detected by a sensor is transmitted to a relay device through a wireless network and then is transmitted from the relay device to a center apparatus through the wireless network.

However, this related-art system requires a wireless network and biological information cannot be acquired out of doors, where the wireless network is not provided. Generally, it is desirable to thin a sensor (reduce the thickness of the sensor from the body surface) for miniaturization and weight reduction. However, if the sensor is miniaturized and is reduced in weight, there is a problem in that a radio wave is absorbed in the living body and a biological signal cannot well be transmitted.

Also in the relay device, a null point where the sensitivity of an antenna vanishes may occur depending on the positional relationship with the sensor or the distance between the antenna and the living body, a state in which a radio signal cannot be received appropriately, and it becomes necessary for the subject to do the same action, exercise, more than once as the position of the relay device is changed, to obtain a biological signal, leading to a heavy burden on the subject.

### SUMMARY

It is therefore an object of the invention to provide a biological information acquisition telemetry system for making it possible to miniaturize and reduce in weight a transmit device for acquiring a biological signal from a living body and acquire stable biological information while the transmit device allows a subject to do action and exercise sufficiently.

In order to achieve the object, according to the invention, there is provided a biological information acquisition telemetry system comprising:

a transmit device, attached to a part of a living body and configured to acquire a biological signal to transmit the biological signal as a radio signal;

a relay device, configured to perform space diversity reception, the relay device comprising:
a first receiver, configured to wirelessly receive the radio signal from the transmit device;
a first transmitter, configured to transmit the radio signal received by the first receiver; and
an attachment unit, adapted to hold the first receiver and the first transmitter and attach the first receiver and the first transmitter to the living body; and

a center apparatus, comprising:
a second receiver, configured to receive the radio signal from the first transmitter of the relay device;
a processor, configured to generate biological information based on the radio signal received by the second receiver; and
a display, configured to display the biological information generated by the processor.

The transmit device may include a sensor configured to acquire the biological signal from the living body.

The transmit device may be integrally provided with the sensor and an electrode for being in contact with a part of the living body.

The relay device may include: two antennas; and spacers for placing the antennas with a distance from the living body.

The transmit device may include an antenna having a loop shape and placed with a distance from the living body.

The first receiver of the relay device may include: a first receiving portion and a second receiving portion, configured to receive the radio signal from the transmit device, respectively; and a processing portion, configured to compare electric field intensity of the radio signal received by the first receiving portion and electric field intensity of the radio signal received by the second receiving portion, and configured to adopt one of the radio signal received by the first receiving portion and the radio signal received by the second receiving portion which has the electric field intensity higher than that of the other.

The first transmitter of the relay device may include an antenna having a shape of a quadratic curve away from a surface of the living body. A tip of the antenna may extend straightly from the surface of the living body.

### BRIEF DESCRIPTION OF THE DRAWINGS

FIG. 1 is a block diagram to show the configuration of a biological information acquisition telemetry system according to the present invention.

FIG. 2 is a functional block diagram of a transmit device included in the biological information acquisition system.

FIGS. 3A, 3B and 3C are drawings to show the appearance of the transmit device; FIG. 3A is a front view; FIG. 3B is a rear view; and FIG. 3C is a bottom view.

FIG. 4 is a sectional side view of the transmit device.

FIG. 5 is a plan view of the transmit device in a state in which a chassis panel on the front of the transmit device is removed.

FIG. 6 is a plan view of an electrode module for an electrocardiogram, connected to the transmit device.

FIG. 7 is a sectional side view to show a transmit device of the type wherein no electrode is included on the back.

FIG. 8 is a plan view of an angle sensor connected to the transmit device.

FIG. 9 is a plan view of a respiratory waveform sensor connected to the transmit device.

FIG. 10 is a plan view of an SpO2 transmit device included in the biological information acquisition telemetry system according to the invention and an SpO2 sensor connected to the SpO2 transmit device.

FIGS. 11A and 11E are perspective views to show the use state of the SpO2 transmit device and the SpO2 sensor.

FIG. 12 is a plan view of a relay device included in the biological information acquisition telemetry system according to the invention.

FIG. 13 is a plan view of the relay device in a state in which a front panel of a chassis of the relay device is removed.

FIG. 14 is a functional block diagram of a part of the relay device.

FIG. 15 is a functional block diagram of a part of the relay device.

FIG. 16 is a front view of the relay device in a state in which an attachment unit is attached to the relay device.

FIG. 17 is a perspective view of the relay device in a state in which a living body wears the relay device to which an attachment unit is attached.

FIG. 18 is a perspective view of the relay device in a state in which a living body wears the relay device to which an attachment unit is attached.

FIG. 19 is a drawing to show the transmission timings of a biological signal transmitted by the biological information acquisition telemetry system according to the invention.

### DETAILED DESCRIPTION OF PREFERRED EMBODIMENTS

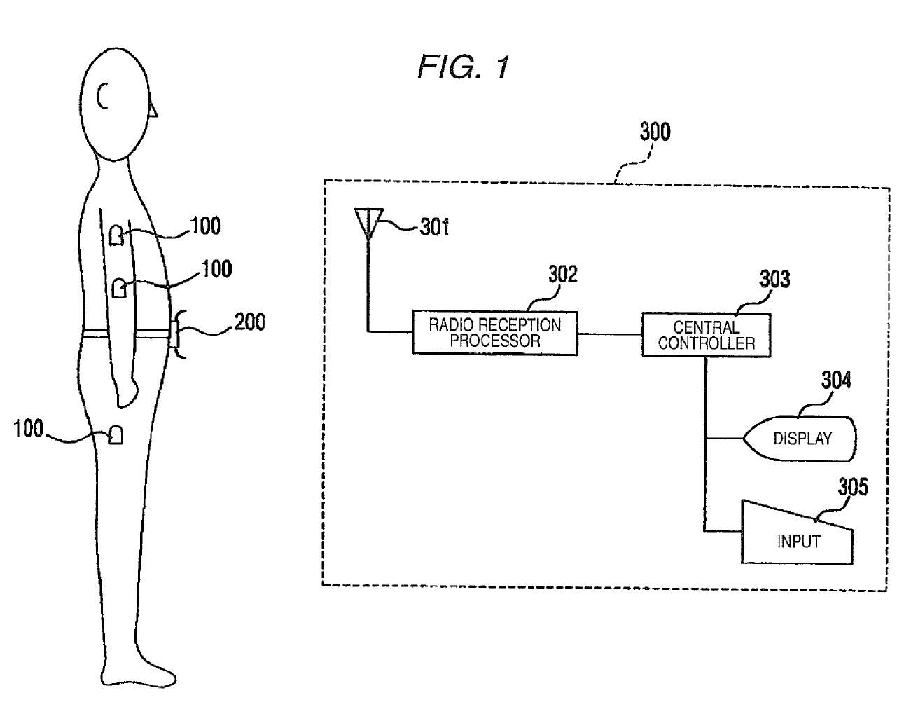

A biological information acquisition telemetry system according to an embodiment of the invention will be discussed with reference to the accompanying drawings. Identical components in the accompanying drawings are denoted by the same reference numerals and duplicate description will not be given. FIG. 1 shows the biological information acquisition telemetry system according to the embodiment of the invention. The biological information acquisition system includes transmit devices 100, a relay device 200, and a center apparatus 300.

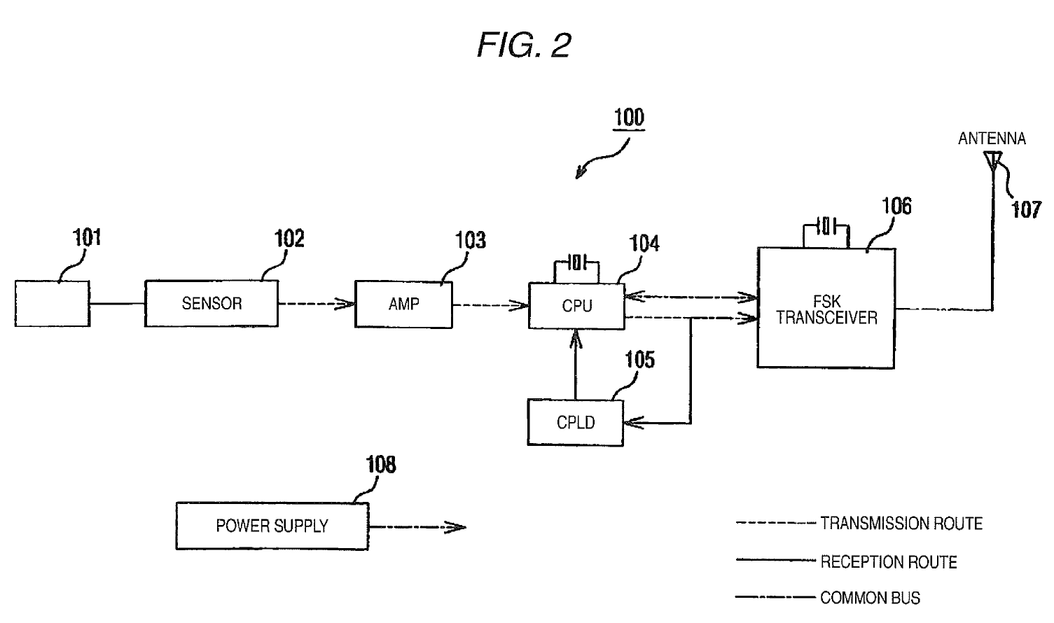

As shown in FIG. 2, the transmit device 100 includes a sensor 102 for acquiring a biological signal by utilizing an electrode 101 to be in contact with a living body. If the transmit device 100 is a transmit device for detecting an electromyogram signal, or the like, the electrode 101 is integral with a chassis.

The transmit device 100 includes an amplifier 103, a CPU (Central Processing Unit) 104, a CPLD (Complex Programmable Logic Device) 105, a FSK (Frequency Shift Keying) transceiver 106, and an antenna (internal lead wire antenna) 107 made of a lead wire. The circuit components receive a power from a power supply 100 having a battery (lithium ion battery) and containing a charging circuit. The amplifier 103 amplifies a biological signal sent from the sensor 102 and feeds the amplified biological signal into the CPU 104. The CPU 104 converts the fed analog biological signal into a digital biological signal and sends the biological signal to the FSK transceiver 106 at a predetermined timing. The CPLD 105 reproduces a clock from a preamble pattern of reception data sent from the relay device 200 and feeds the clock into the CPU 104 for detecting the transmission timing. Here, TDMA (Time Division Multiple Access) is adopted and transmission is executed according to a preset time slot.

The FSK transceiver 106 FSK-modulates sampling data of the biological signal sent from the CPU 104, upconverts the signal into a predetermined radio frequency (medical radio E band), and transmits the signal from the antenna 107. The FSK transceiver 106 also receives a radio signal sent from the relay device 200 through the antenna 107, downconverts the signal, and sends the signal to the CPLD 105. The FSK transceiver 106 can use two frequencies and transmits according to the setup one frequency.

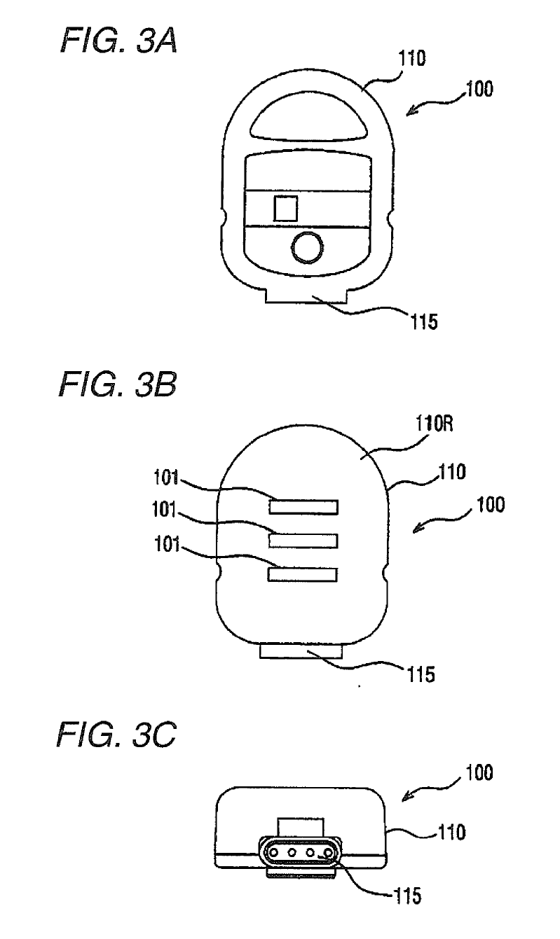

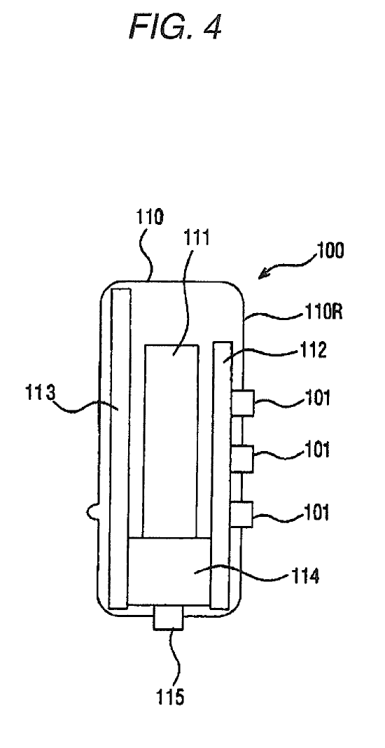

The transmit device 100 has the circuits, etc., housed in a chassis 110 shaped like a plane provided by joining a semicircle to a quadrangle as shown in FIGS. 3A to 3C and 4. In the chassis 110, a rechargeable battery 111 is placed at the center and an analog unit board 112 including the sensor 102, the amplifier 103, the power supply 108, etc., is placed in the portion to the living body side from the battery 111 at the operating time. Further, an RF-CPU unit board 113 including the CPU 104, the CPLD 105 and the FSK transceiver 106 is placed on opposite side to the analog unit board 112 with the battery 111 between.

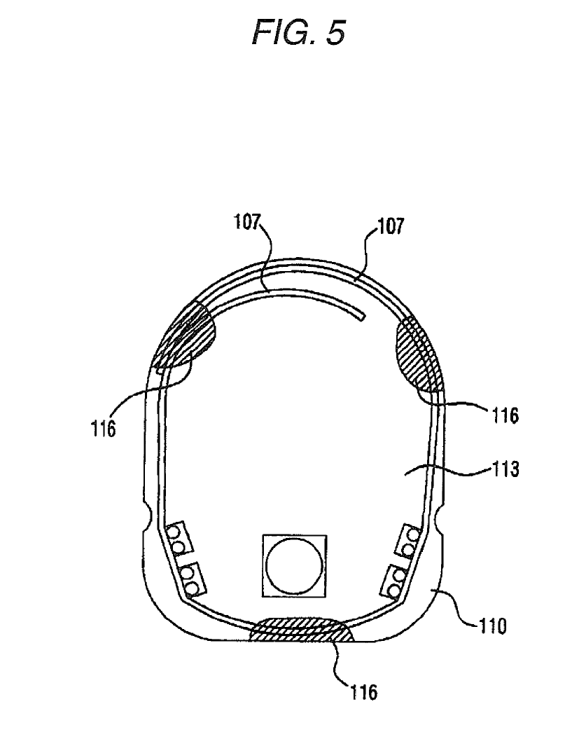

A board connection connector 114 for connecting the analog unit board 112 and the RF-CPU unit board 113 is placed therebetween. Further, an external connection connector 115 for connecting the electrode 101 provided on the outside of the chassis 110 is provided. Two antennas 107 each having a length of a quarter wavelength relative to a useful frequency are shaped like a loop along the margin of the chassis 110 on the top of the REF-CPU unit board 113, the side at a distance from the living body, as shown in FIG. 5, which is a plan view of the transmit device 100 with the RF-CPU unit board 113 side of the chassis 110 opened, and the transmission distance is enhanced although the two antennas 107 are whip antennas. The two antennas 107 are fixed with an adhesive 116 at appropriate positions.

There are types of transmit devices 100 having sensor functions for an electromyogram, an electrocardiogram, acceleration (uniaxial and triaxial), a DC (external input), an angle, a respiratory waveform, SpO2 (oxygen saturation of arterial blood), etc., and the electrode 101 and the sensor 102 are changed corresponding to each of the types.

The transmit device 100 shown in FIGS. 3A to 3C and 4 is the type having the electromyogram sensor function, and three electrodes 101 project on a back 110R of the chassis 110. To use the transmit device 100, double-side tape of the same shape as the back 110R of the chassis 110, formed with holes corresponding to the three electrodes 101 is attached on the back 110R, the required parts of a living body are wiped with alcohol, and the back 110R of the chassis 110 is attached on the living body with the double-side tape. The sensor 102 includes a related-art configuration for an electromyogram.

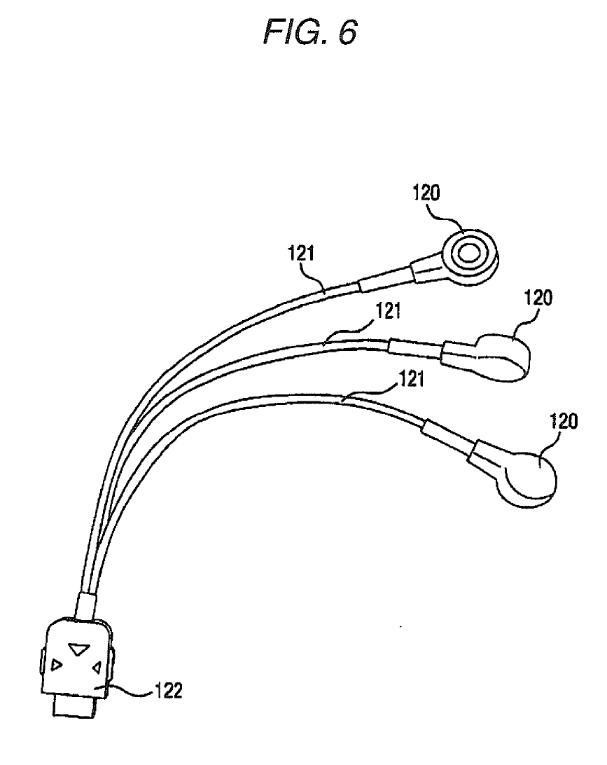

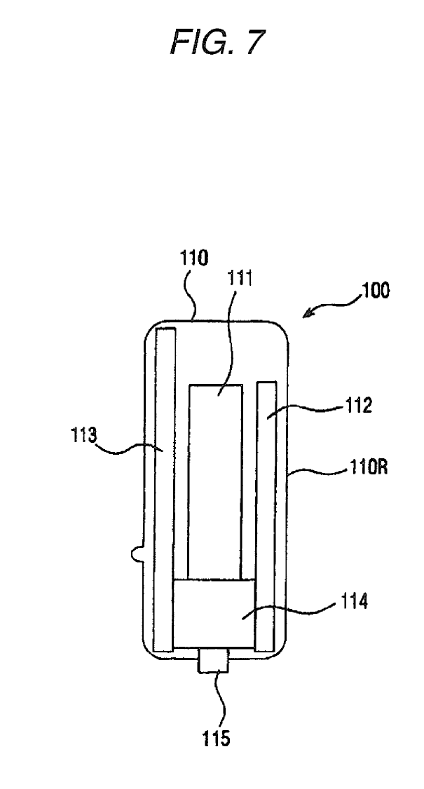

The transmit device 100 for an electrocardiogram uses an electrode module provided by connecting three electrode terminals 120 to separate lead wires 121 and connecting the lead wires 121 to a plug 122 as shown in FIG. 6. The plug 122 is joined to the external connection connector 115 of the chassis 110, the three electrode terminals 120 are jointed to the three electrodes (living body electrodes) 101 each having a conductive gel, etc., and the electrodes 101 are attached on the required positions of the chest of a living body. The transmit device 100 for an electrocardiogram is not provided with the electrodes 101 on the back 110 of the chassis 110 and has an almost flat face as shown in FIG. 7. Double-side tape of the same shape as the back 110R of the chassis 110 is put on the back 110R, the required parts of a living body are wiped with alcohol, and the back 110R of the chassis 110 is put on the living body with the double-side tape. The sensor 102 includes a related-art configuration for an electrocardiogram.

The transmit device 100 for acceleration (uniaxial and triaxial) includes a related-art sensor 102 containing an acceleration detection mechanism in the chassis 110. An electrode for coming in contact with a living body does not exist. The transmit device 100 is not provided with the electrodes 101 on the back 110R of the chassis 110 either and has a structure as shown in FIG. 7. To use the transmit device 100, double-side tape of the same shape as the back 110R of the chassis 110 is put on the back 110R, the required parts of a living body are wiped with alcohol, and the back 110R of the chassis 110 is attached on the living body with the double-side tape.

The transmit device 100 for a DC introduces an external signal into the external connection connector 115 with an input cord and does not include the sensor 102. An electrode for coming in contact with a living body does not exist. The back 110R of the chassis 110 is as shown in FIG. 7. The use method is similar to that of the transmit device 100 for acceleration (uniaxial and triaxial).

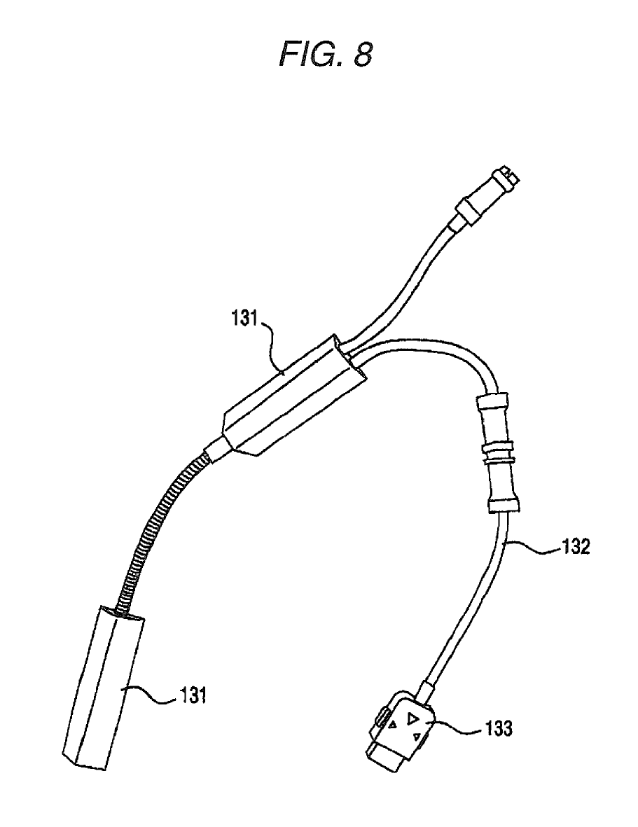

The transmit device 100 for an angle has two detectors 131 as shown in FIG. 8 and uses a sensor to send a signal from the detector 131 through a lead wire 132 to a plug 133. The plug 133 is joined to the external connection connector 115 of the chassis 110 and the two detectors 131 are put up and down or from side to side through a joint, whereby measurement is conducted. The transmit device 100 for an angle does not have an electrode for coming in contact with a living body. The back 110R of the chassis 110 is as shown in FIG. 7. To use the transmit device 100, double-side tape of the same shape as the back 110R of the chassis 110 is put on the back 110R, the required parts of a living body are wiped with alcohol, and the back 110R of the chassis 110 is put on the living body with the double-side tape.

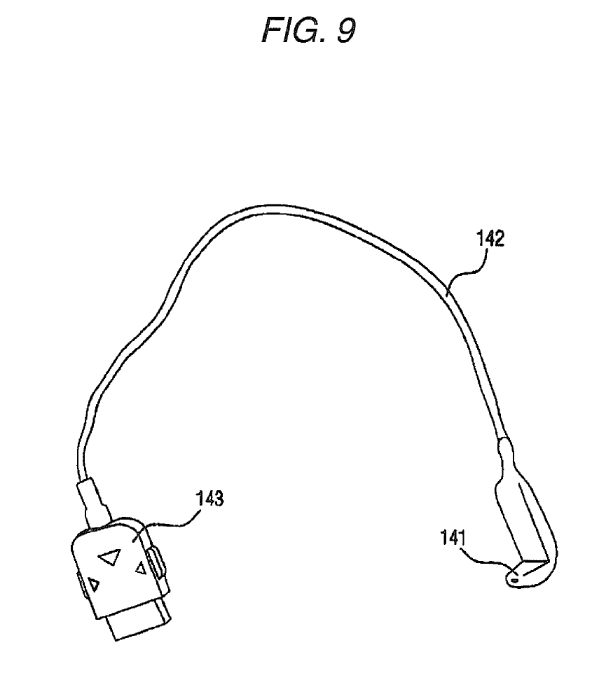

The transmit device 100 for a respiratory waveform is provided with a detector 141 of a thermistor as shown in FIG. 9, for example, and uses a sensor to send a signal from the detector 141 through a lead wire 142 to a plug 143. The sensor 102 takes out a signal for temperature change provided by the detector 141 of the thermistor. The transmit device 100 for a respiratory waveform does not have an electrode for coming in contact with a living body. The back 110R of the chassis 110 is as shown in FIG. 7. To use the transmit device 100, the detector 141 of the thermistor is fixed to the entrance of a nostril and the lead wire 142 is fixed to the face, etc., as required. Double-side tape of the same shape as the back 110R of the chassis 110 is put on the back 110R, the required parts of a living body are wiped with alcohol, and the back 110R of the chassis 110 is put on the living body with the double-side tape.

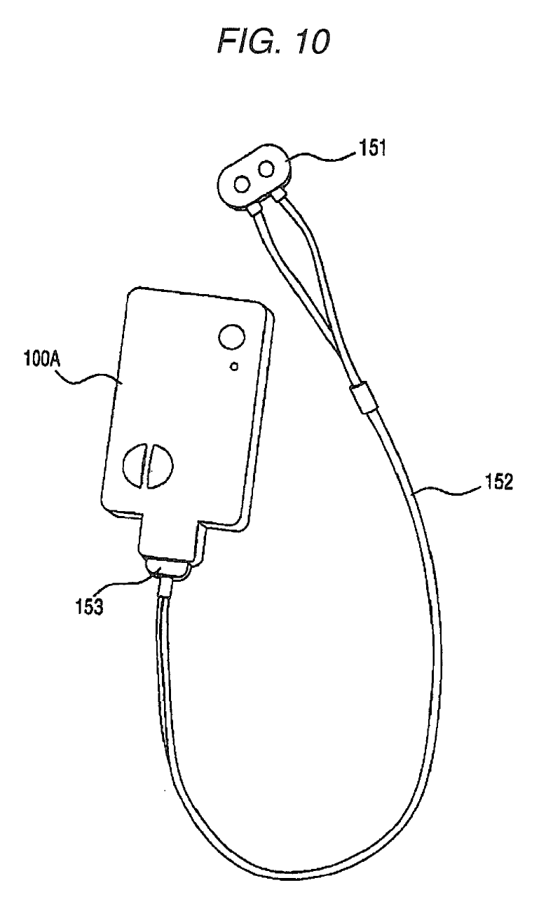

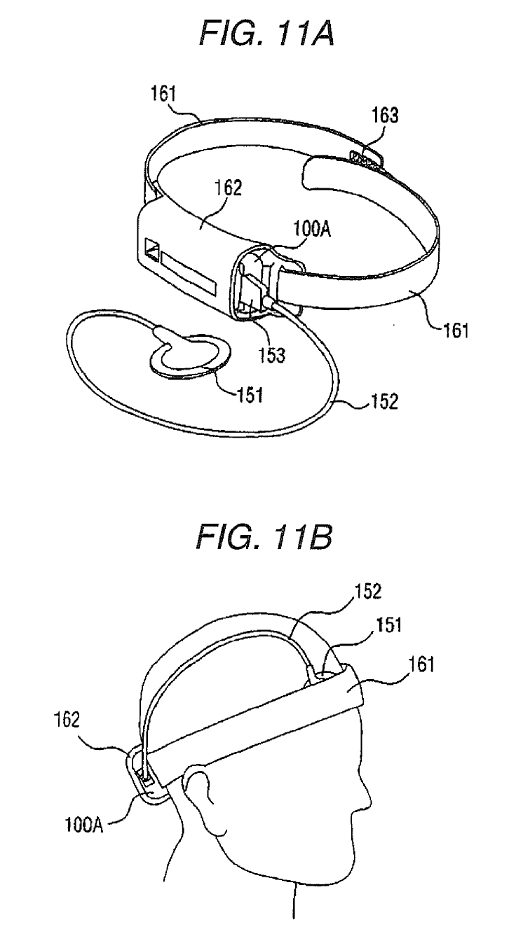

A sensor including a detector 151 having a light receiving element and a light transmitting element connected to a plug 153 through a lead wire 152 is connected to an external connection connector 115 of a transmit device 100A for SpO2, for example, as shown in FIG. 10. The internal configuration of the transmit device 100A is as shown in FIG. 2. A related-art configuration for SpO2 is used as a sensor 102. The transmit device 100A is housed in a housing case 162 attached to a head band 161 as shown in FIGS. 11A and 11B for use. The detector 151 is attached in the vicinity of the center of a forehead and is firmly bound with the head band 161. A sheet fastener 163 is provided over a predetermined length from both ends of the headband 161, and the transmit device 100A housed in the housing case 162 is fixed to the back of a head, for example (FIG. 11B).

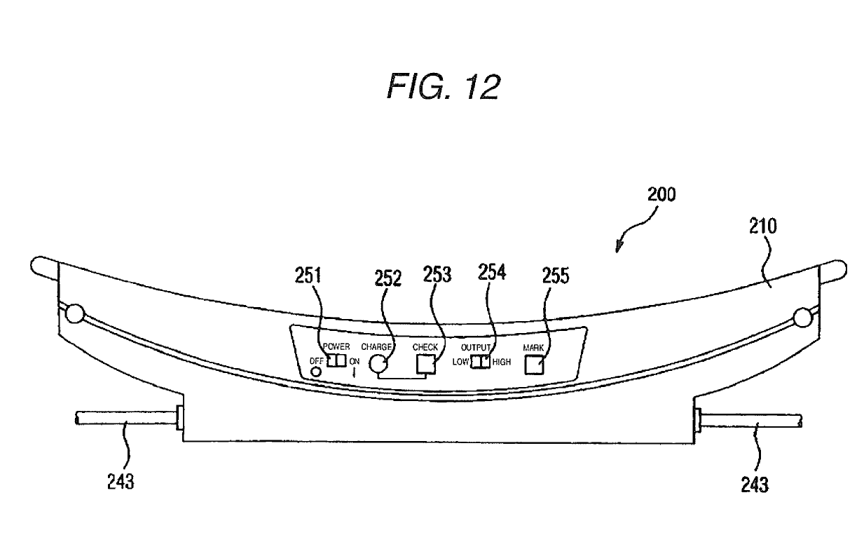

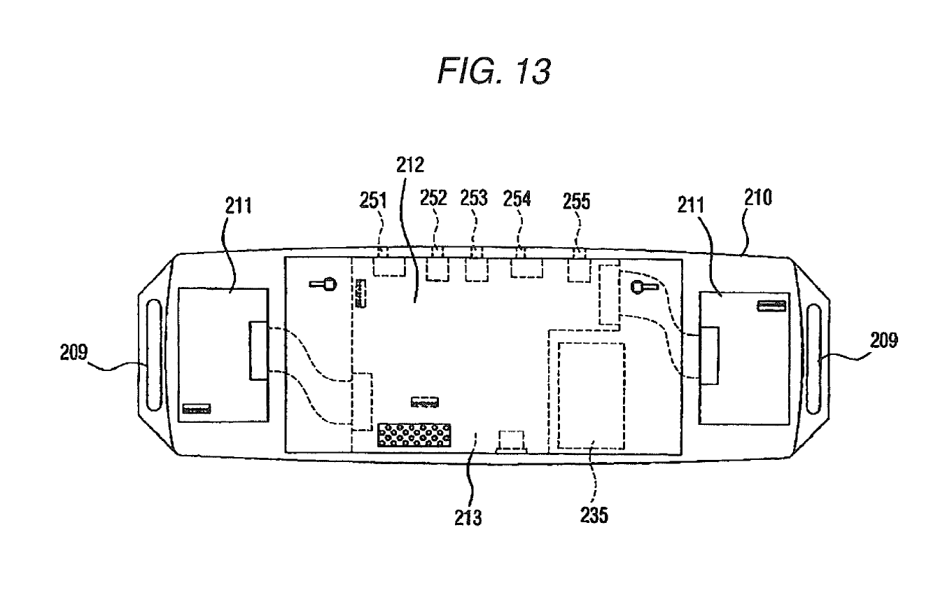

The relay device 200 has a back along an R shape so as to be fitted to the abdomen or the lumbar area of a living body as shown in FIG. 12. In the center of the surface of the relay device 200, two transmission boards 211 and a transmission-reception radio board 212 and a transmission-reception control board 213 of a two-layer structure are included in a box-like chassis 210 made flat, as shown in FIG. 13.

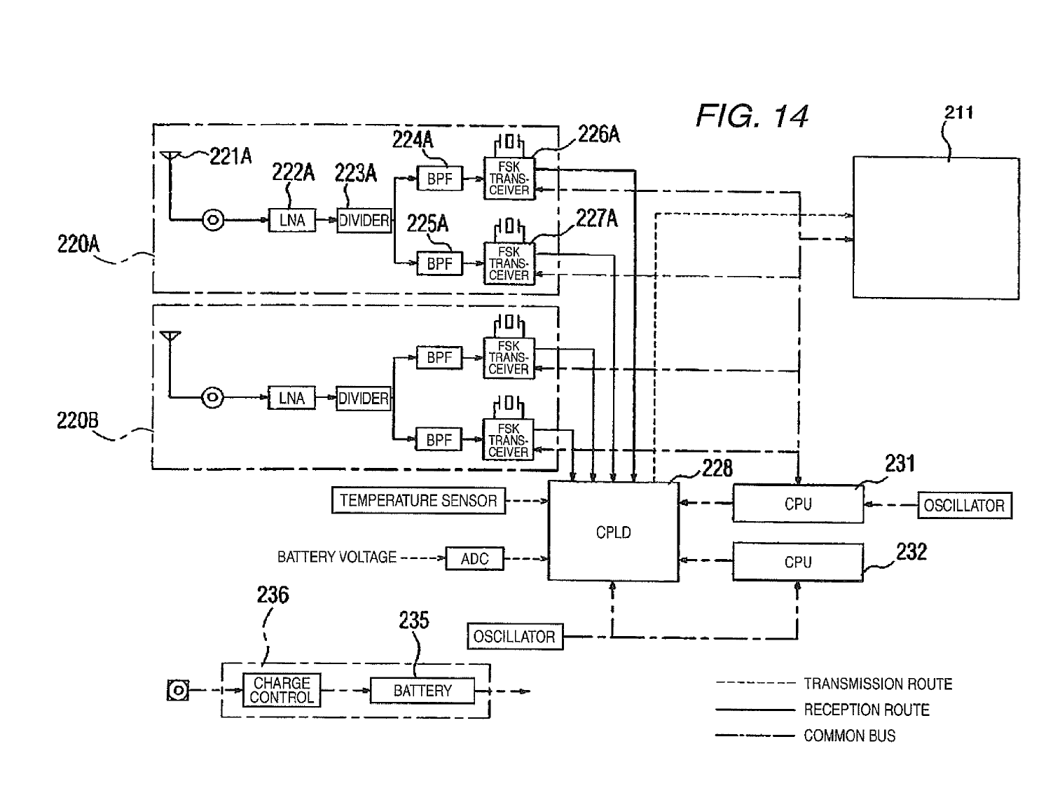

The circuit configuration of the transmission-reception radio board 212 and the transmission-reception control board 213 is as shown in FIG. 14. The relay device 200 includes a first receiver 220A and a second receiver 220B for a space diversity reception technique. The first receiver 220A and the second receiver 220B are of the same configuration and therefore the first receiver 220A will be discussed as a representative. The first receiver 220A includes a reception antenna 221A of a λ/4 dipole antenna made of a lead wire and a low-noise amplifier 222A and inputs and amplifies a radio signal.

Output of the low-noise amplifier 222A is branched to BPFs (band pass filters) 224A and 225A by a divider 223A. The BPFs 224A and 225A correspond to two used radio frequencies and allow different predetermined frequency components to pass through.

Outputs of the BPFs 224A and 225A are fed into FSK transceivers 226A and 227A and are downconverted and FSK-demodulated and are sent to a CPLD 228. The CPLD 228 performs processing of comparing the electric field intensities of the reception signals received from the first receiver 220A and the second receiver 220B and adopting the reception signal of the higher electric field intensity and in addition, reproducing a reception clock from a preamble pattern of the reception signal, etc.

A first CPU 231 and a second CPU 232 are connected to the CPLD 228. The first CPU 231 performs operation control of the 5 elements in the relay device 200 and the second CPU 232 performs TDMA reception processing in cooperation with the CPLD 228. Here, for example, dual partitioning of eight time slots about one frequency is executed; a signal can be received from eight transmit devices 100 about one frequency and with two frequencies, a signal can be received from 16 transmit devices 100.

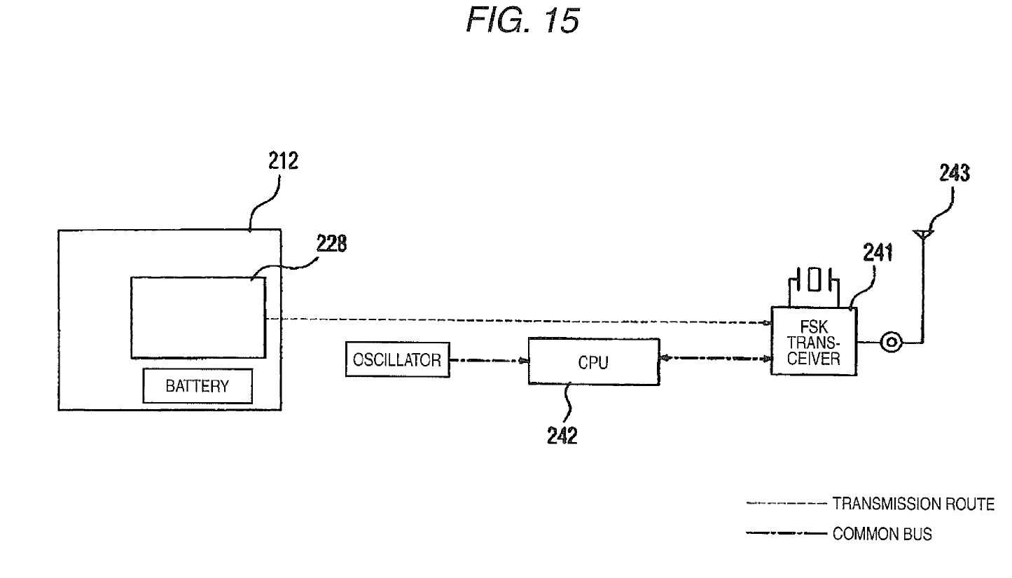

The circuit components receive a power from a power supply 236 having a battery (lithium ion battery) 235. The signal received from the CPLD 228 and a receiver synchronous pattern and a status indicating the state of the apparatus to be set in a preamble pattern are sent to the two transmission boards 211. Transmission circuits provided in the two transmission boards 211 are of the same configuration and are shown in FIG. 15 although they differ in used transmission frequency.

That is, the signal sent from the CPLD 228 arrives at an FSK transceiver 241 and a CPU 242 controls the FSK transceiver 241 so as to transmit signals received from the eight transmit devices 100 by conducting TDMA communications. The FSK transceiver 241 performs FSK modulation and up-conversion and each signal is transmitted from a transmission antenna 243 of a λ/4 dipole antenna made of a lead wire.

The relay device 200 is provided on a top board with a power switch 251, a check LED 252, a check switch 253, a transmission power changeover switch 254, and a mark switch 255 as shown in FIG. 12. The user can know an operable state by turning on the power switch 251 and operating the check switch 253 to light the check LED 252 green. The mark switch 255 is operated, whereby a reception event can be caused to occur in an operation state for executing reception.

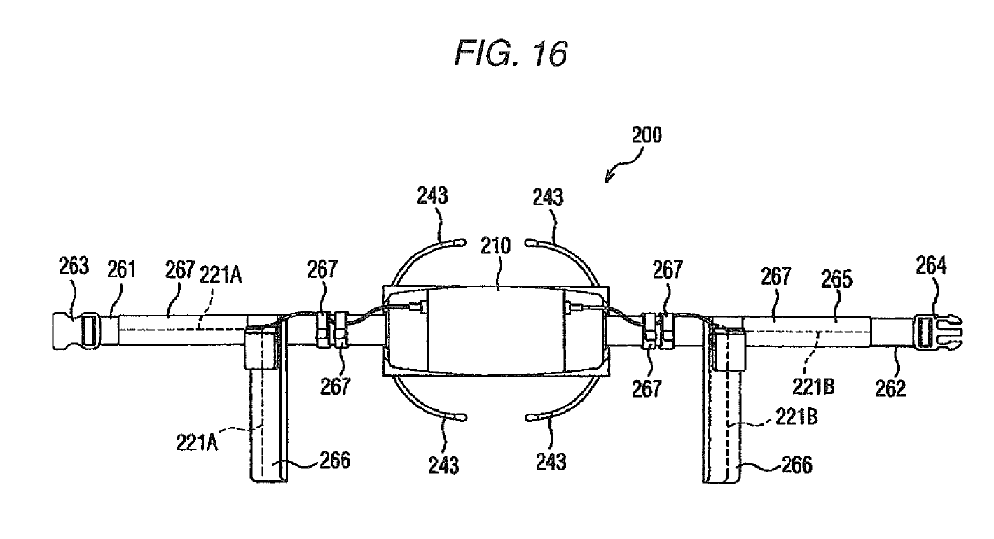

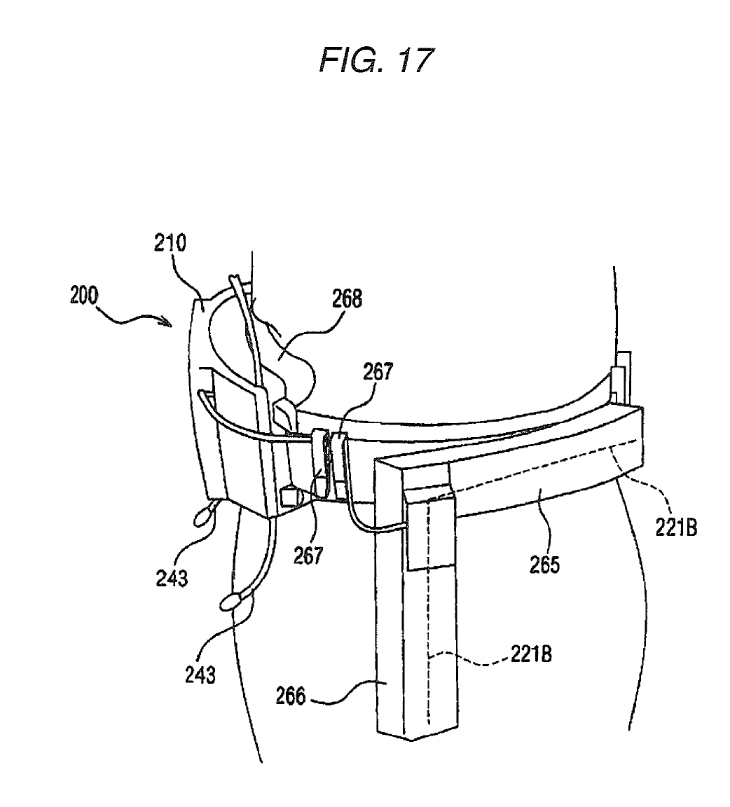

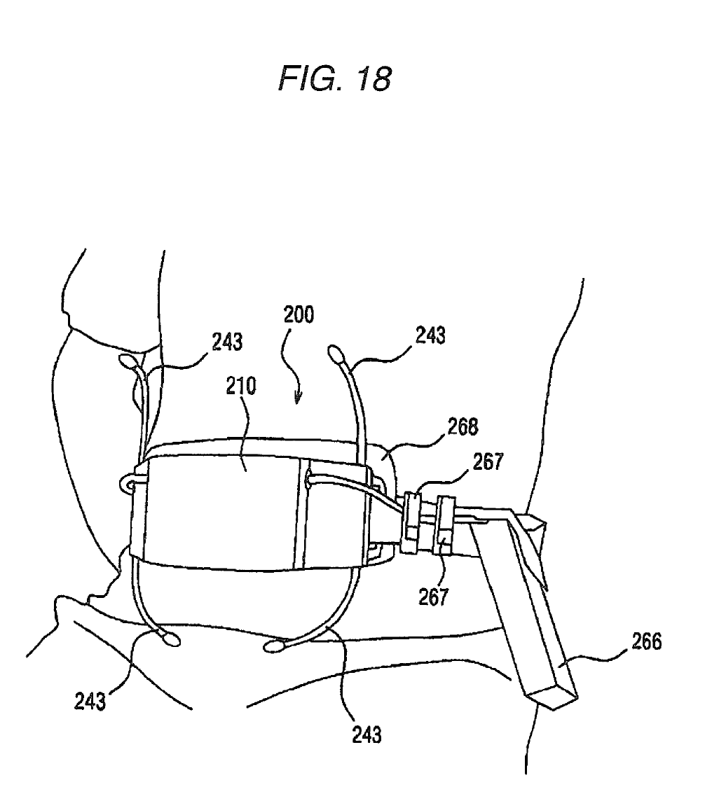

The chassis 210 of the relay device 200 is formed on both sides with belt holes 209 shown in FIG. 13 and belts 261 and 262 are attached to the belt holes 209 as shown in FIGS. 16 to 18. A reception stopper 263 and an insertion stopper 264 each having a length adjustment part are provided at the tips of the belts 261 and 262. The belts 261 and 262 are provided with L-shaped antenna bags 265. Each of the antenna bags 265 is a bag with a corner of the L letter formed as an entrance, and a spacer 266made of an elastic body of sponge, etc., is contained in the bag. When a living body wears the relay device 200, the reception antenna 221A, 221B is opposed to the surface of the living body with the spacer 266 between so that the reception antenna is placed with a distance from the surface of the living body. It was acknowledge by experiment that if the spacer 266 is more than 3 cm in thickness, preferred reception is made possible.

Each of the belts 261 and 262 is provided with a belt hook 267 for fixing an antenna cord for connecting the reception antenna 221A, 221B and the low-noise amplifier 222A, 222B. Two transmission antennas 243 connected to the two transmission boards 211 project from both sides of the chassis 210 of the relay device 200 and are bent like a quadratic curve away from the surface of the living body with the tip of each antenna extending straightly from the surface of the living body. Accordingly, the degree of absorption of a transmitted radio signal in the living body is lowered and good transmission is made possible. A cushion 268 is put on the back of the relay device 200. When a living body wears the relay device 200, the cushion 268 is fitted to the living body and prevents large swinging caused by exercise, etc. The relay device 200 can also be used as it is put on the abdomen or the lumbar area of a living body as shown in FIG. 13; the relay device 200 can also be used as it is removed from a living body with the belts 261 and 262 extended as shown in FIG. 16.

As shown in FIG. 1, the center apparatus 300 includes an antenna 301, a radio reception processor 302, a central controller 303, a display 304, and an input 305. The radio reception processor 302 receives a signal corresponding to two transmission frequencies of the relay device 200, receives a TDMA communication signal, FSK-demodulates the signal to reproduce a biological signal corresponding to each time slot, and feeds the biological signal into the central controller 303.

The central controller 303 is a processor for processing the data of the biological signal, generating an electromyogram waveform, an electrocardiogram waveform, an acceleration waveform (uniaxial and triaxial), a DC waveform, an angle waveform, a respiratory waveform, an SpO2 waveform, etc., and displaying each waveform on the display 304. The user can enter a command such as a display switch command given to the central controller 303 through the input 305. The elements except the antenna 301 or the radio reception processor 302 can also be implemented as a personal computer, etc.

In the biological information acquisition telemetry system described above, for example, 16 transmit devices for an electromyogram are provided as the transmit devices 100, the eight transmit devices are set to those using a first frequency, and the remaining eight transmit devices are set to those using a second frequency. The time slots are allocated so that the time slots used for the eight transmit devices 100 using the first frequency do not overlap. The time slots are allocated so that the time slots used for the eight transmit devices 100 using the second frequency do not overlap.

The transmit devices 100 are each powered on and are attached on the required parts of a living body as previously described. The relay device 200 with power on is set on the abdomen of the living body and measurement is started. Of course, the center apparatus 300 is also powered on and is placed in an operable state

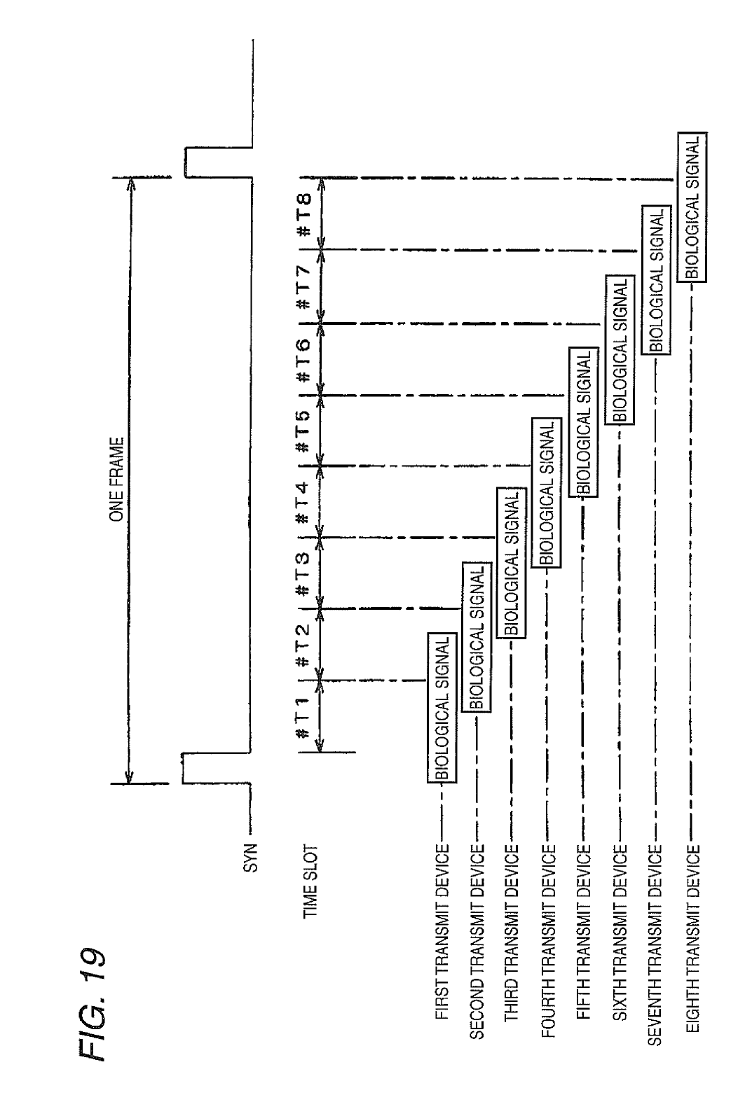

Each of the transmit devices 100 reproduces a clock from a preamble pattern contained in a transmission signal sent from the relay device 200 and detects the timing (position) of a synchronous signal at the top of the eight time slots. The synchronous signal SYN is reproduced as shown in FIG. 19. Since the time slots are allocated to the transmit devices 100, each of the transmit devices 100 FSK-modulates the acquired biological signal for transmission at the predetermined manieth time slot from the pulse of the synchronous signal SYN. The biological signal is sent from each of the corresponding transmit devices 100 at time slots #T1, #T2, . . . , #T8 in FIG. 19.

The relay device 200 performs diversity reception of a coming radio signal, reproduces a reception clock from the preamble pattern of the reception signal, takes out the biological signal at each time slot, restores the signal to one frame of eight time slots, and adds a receiver synchronous pattern and a status indicating the state of the apparatus and transmits.

The center apparatus 300 receives the biological signal placed at the eight time slots according to two frequencies, takes out the biological signal from the time slots using the receiver synchronous pattern, processes the data of the taken-out biological signal to generate an electromyogram waveform in response to the biological signal, and displays the electromyogram waveform on the display 304.

Thus, according to the embodiment, the biological signal is transmitted from the transmit device 100 to the relay device 200, whereby the subject can do exercise, etc., as desired in a state in which the subject wears the transmit device 100 and the relay device 200, and biological information in a moving state can be acquired. In this case, the two antennas 107 of the transmit device 100 are shaped like a loop along the margin of the chassis 110 on the top of the RE-CPU unit board 113, the side at a distance from the living body, and the transmission distance is enhanced for contributing to reliable transmission of the biological signal.

In the relay device 200, space diversity reception is performed, the reception antenna 221A, 221B is opposed to the surface of the living body with the spacer 266 between so that the reception antenna is placed with a distance from the surface of the living body to enable preferred reception, and biological information can be reliably acquired. Further, the TDMA communication system is adopted, so that the biological signals can be acquired at a time from the transmit devices 100, the biological signals in the parts or the biological signals according to a plurality of parameters can be obtained, and the activity state of the living body can be analyzed.

In the biological information acquisition telemetry system according to an aspect of the invention, the biological signal acquired by the sensor is transmitted as a radio signal from the transmit device, the radio signal is received in the relay device according to the space diversity reception system, and the received signal is relayed and transmitted through the radio line, so that the receiver sensitivity of the signal transmitted from the transmit device in the relay device becomes high and it is made possible to acquire biological information appropriately.

In the biological information acquisition telemetry system according to an aspect of the invention, the transmit device includes the electrode for coming in contact with the living body and the sensor in one piece, so that noise to the biological signal obtained from the electrode does not superpose through the lead wire and can be decreased and stable biological information can be acquired.

In the biological information acquisition telemetry system according to an aspect of the invention, the relay device further includes two antennas for performing space diversity reception and the spacer for placing the antennas with a distance from the living body, so that it is made possible to receive a radio signal and stable biological information can be acquired.

In the biological information acquisition telemetry system according to an aspect of the invention, the antenna provided with the transmit device is shaped like a loop on the side with a distance from the living body, so that a transmission radio wave is hard to absorb in the living body and stable biological information can be acquired.

What is claimed is:

1. A biological information acquisition telemetry system comprising:
a transmit device, attached to a part of a living body and configured to acquire a biological signal to transmit the biological signal as a radio signal;
a relay device, configured to perform space diversity reception, the relay device comprising:
a first receiver, configured to wirelessly receive the radio signal from the transmit device;
a first transmitter, configured to transmit the radio signal received by the first receiver; and
an attachment unit, adapted to hold the first receiver and the first transmitter and attach the first receiver and the first transmitter to the living body; and
a center apparatus, comprising:
a second receiver, configured to receive the radio signal from the first transmitter of the relay device;
a processor, configured to generate biological information based on the radio signal received by the second receiver; and
a display, configured to display the biological information generated by the processor.

2. The biological information acquisition telemetry system as claimed in claim 1, wherein
the transmit device includes a sensor configured to acquire the biological signal from the living body.

3. The biological information acquisition telemetry system as claimed in claim 2, wherein
the transmit device is integrally provided with the sensor and an electrode for being in contact with a part of the living body.

4. The biological information acquisition telemetry system as claimed in claim 1, wherein
the relay device includes:
two antennas; and
spacers for placing the antennas with a distance from the living body.

5. The biological information acquisition telemetry system as claimed in claim 1, wherein
the transmit device includes an antenna having a loop shape and placed with a distance from the living body.

6. The biological information acquisition telemetry system as claimed in claim 1, wherein
the first receiver of the relay device includes:
a first receiving portion and a second receiving portion, configured to receive the radio signal from the transmit device, respectively; and
a processing portion, configured to compare electric field intensity of the radio signal received by the first receiving portion and electric field intensity of the radio signal received by the second receiving portion, and configured to adopt one of the radio signal received by the first receiving portion and the radio signal received by the second receiving portion which has the electric field intensity higher than that of the other.

7. The biological information acquisition telemetry system as claimed in claim 1, wherein
the first transmitter of the relay device includes an antenna having a shape of a quadratic curve away from a surface of the living body, and
a tip of the antenna extends straightly from the surface of the living body.
